(19) United States
(12) Patent Application Publication
(10) Pub. No.: US 2022/0071560 A1
(43) Pub. Date: Mar. 10, 2022

(54) METHOD AND SYSTEM FOR GENERATING A BIOMARKER QUANTIFYING SPATIAL HOMOGENEITY OF A MEDICAL PARAMETER MAP

(71) Applicant: Siemens Healthcare GmbH, Erlangen (DE)

(72) Inventor: Robert Grimm, Nuernberg (DE)

(73) Assignee: Siemens Healthcare GmbH, Erlangen (DE)

(21) Appl. No.: 17/468,891

(22) Filed: Sep. 8, 2021

(30) Foreign Application Priority Data
Sep. 8, 2020 (DE) 102020211271.1

(51) Int. Cl. A61B 5/00; A61B 5/026; A61B 5/055; G01R 33/44; G16H 30/40; G16H 50/30

(52) U.S. Cl. CPC A61B 5/4842; A61B 5/4848; A61B 5/0263; A61B 5/4872; G16H 50/30; A61B 5/0035; G01R 33/448; G16H 30/40; A61B 5/055

(57) ABSTRACT

In a method for generating a biomarker quantifying spatial homogeneity of a medical parameter map, A) the parameter map is provided, B) at least two parameter classes are provided, where each parameter value is to be assigned to a parameter class, C) a sub-area of the examination area is selected, D) in each case, a frequency value for each parameter class for the sub-area is determined, E) a heterogeneity indicator for the sub-area is determined based on the frequency values of the parameter classes, F) further heterogeneity indicators are generated for at least two further sub-areas that are at least partially different from one another, G) a statistical homogeneity value is determined by statistical evaluation of the heterogeneity indicator of the sub-area and the further heterogeneity indicators, and H) the statistical homogeneity value is provided as a biomarker.

### CROSS REFERENCE TO RELATED APPLICATIONS

This patent application claims priority to German Patent Application No. 10 2020 211 271.1, filed Sep. 8, 2020, which is incorporated herein by reference in its entirety.

### BACKGROUND

### Field

The disclosure relates to a method, an image evaluator, a computer program product and an electronically readable data carrier for generating a biomarker.

### Related Art

Medical parameter maps are typically based on spatially resolved medical data from an examination area of a subject of the examination. The examination area is typically the area of the subject of the examination from which medical data has been acquired with a spatial resolution, for example by means of a magnetic resonance device. Medical parameter maps typically comprise quantitative parameter values in spatial resolution from the examination area for a parameter. The quantitative parameter values can be continuous. The parameter can characterize physical properties, in particular of molecules or atoms present in the examination area, such as, for example, relaxation parameters, density, elasticity, mobility. The quantitative parameter values can be discrete, i.e. assigned to specific parameter classes. In particular, the parameter can characterize a tissue property and/or a tissue and/or cells. The parameter can in particular comprise cell types.

Medical parameter maps can accordingly be used to establish a diagnosis and/or to check the effectiveness of therapy for the subject of the examination. The homogeneity of the parameter can be an indicator of a disease and/or a pathological phenomenon and/or the effectiveness of therapy for the subject of the examination. To date, in particular in the field of tumor monitoring, evaluations of the homogeneity or heterogeneity of parameter maps have been performed qualitatively by a radiologist, supported by color classification and the depiction of different tumor classes. Quantitative evaluations of parameter maps are in particular known in the field of volumetry, wherein here, however, the homogeneity and/or heterogeneity of the classified parameter is irrelevant.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a flowchart of a method according to an exemplary embodiment of the disclosure.

FIG. 2 is a flowchart of a method according to an exemplary embodiment of the disclosure.

FIG. 3 illustrates a selection of a sub-area using a binary property map in a schematic depiction, according to an exemplary embodiment of the disclosure.

FIG. 4 illustrates an example for ascertaining a statistical homogeneity value for a first parameter map, according to an exemplary embodiment of the disclosure.

FIG. 5 illustrates an example for ascertaining a statistical homogeneity value for a second parameter map, according to an exemplary embodiment of the disclosure.

FIG. 6 illustrates an example for ascertaining a statistical homogeneity value for a third parameter map, according to an exemplary embodiment of the disclosure.

FIG. 7 illustrates an image evaluator according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to provide a method for generating a particularly simple and robust quantitative biomarker representative of the spatial homogeneity of a medical parameter map.

The method according to an exemplary embodiment of the disclosure for generating a biomarker quantifying spatial homogeneity of a medical parameter map, wherein the parameter map in each case has a parameter value for a plurality of voxels mapping an examination area, provides the following method steps:
A) providing the parameter map,
B) providing at least two parameter classes, wherein each parameter value is to be assigned to a parameter class,
C) selecting a sub-area of the examination area,
D) determining in each case a frequency value for each parameter class for the sub-area, said frequency value in each case indicating the proportion of the parameter values occurring in this parameter class in the sub-area,
E) determining a heterogeneity indicator for the sub-area taking into account the frequency values of the parameter classes,
F) generating further heterogeneity indicators for at least two further sub-areas that are at least partially different from one another by repeating steps C), D), E) for the at least two further sub-areas,
G) ascertaining a statistical homogeneity value by statistical evaluation of the heterogeneity indicator of the sub-area and the further heterogeneity indicators of the further sub-areas,
H) providing the statistical homogeneity value as a biomarker.

The parameter map typically comprises in each case one parameter value for a voxel. The voxels are typically arranged according to the spatial resolution of the examination area. Each voxel typically comprises precisely one section of the examination area.

The provision of the parameter map can comprise the generation of the parameter map. The generation of the parameter map can take place by processing medical image data. The generation of the parameter map can comprise an analysis of medical data recorded by means of medical imaging devices. The provision of the parameter map can comprise recording medical data and/or processing the medical data.

The parameter values of the parameter map are typically within a value range. The parameter classes are typically embodied such that the value range of the parameter values of the parameter map can be disjunctively divided into at least two parameter classes. The at least two parameter classes are typically embodied such that each voxel can be and/or is uniquely assigned to a parameter class on the basis of the corresponding parameter value. In particular, the at least two parameter classes can be selected such that these parameter values indicate different diagnoses and/or diseases and/or mutations and/or cell states and/or pathological changes. The parameter classes are preferably defined in advance. The parameter classes are preferably based on a relationship between the parameter values and a disease and/or a course of therapy.

The sub-area can be selected by selecting voxels on the parameter map. The sub-area can be selected according to a prespecified pattern. The sub-area preferably comprises a defined number of voxels. The sub-area is preferably embodied as rectangular or cuboid. The sub-area can, for example, comprise 5×5×5 or 5×5×3 voxels.

The determination of a frequency value for each parameter class for the sub-area can comprise a classification of the parameter values occurring within the sub-area into the at least two parameter classes.

The determination of a frequency value in each case preferably takes place for each parameter class of the at least two parameter classes for the sub-area. The frequency value for a parameter class of the at least two parameter classes preferably comprises the relative proportion of the parameter values occurring in this parameter class in the sub-area in relation to all the voxels comprised by the sub-area. The number of frequency values determined typically corresponds to the number of parameter classes provided.

The determination of the heterogeneity indicator for the sub-area preferably takes place taking into account all the frequency values determined for the sub-area.

The method according to an exemplary embodiment of the disclosure provides for the selection of at least two further sub-areas. The number of further sub-areas is preferably selected such that each voxel of the parameter map is comprised by at least the sub-area or a further sub-area. Apart from the voxels at the edge of the parameter map, the number of further sub-areas comprising a voxel preferably corresponds to the number of voxels within a further sub-area. The size of the further sub-areas preferably corresponds to the size of the sub-area. The sub-area and the further sub-areas preferably differ by at least one voxel. If the sub-area is cuboid or rectangular, at least one further sub-area typically differs by voxels of a row and/or a column. The method according to the disclosure provides that for each of the at least two further sub-areas in each case a further frequency value is determined for each parameter class and from this a further heterogeneity indicator. This is typically followed by a statistical evaluation of the resulting at least three heterogeneity indicators and thus the ascertainment of the statistical homogeneity value. The statistical evaluation of the heterogeneity indicators of the different sub-areas can, for example, comprise the formation of a mean value and/or a median. The statistical evaluation of the heterogeneity indicators of the different sub-areas can comprise the formation of a minimum value, a maximum value and/or a percentile. The statistical evaluation of the heterogeneity indicators of the different sub-areas can comprise the determination of a proportion of the heterogeneity indicators which exceed and/or fall below a threshold value.

One of the advantages of the method according to an exemplary embodiment of the disclosure is that the biomarker determined as a single value can characterize local homogeneities or heterogeneities of the parameter map and is thus simple to understand. It is also conceivable for two or more statistical homogeneity values to be determined based on heterogeneity indicators ascertained in different ways in the context of the method according to the disclosure. The biomarker can comprise these two or more statistical homogeneity values. The statistical homogeneity values and/or heterogeneity indicators ascertained in different ways can differ in their sensitivity with respect to the locality and/or heterogeneity and/or significance of individual parameter classes. Thus, the biomarker can be used more extensively and informatively.

The granularity of the biomarker is preferably dependent on the size of the selected sub-areas as a result of which the granularity can be set in dependence on the purpose of the biomarker and in particular in dependence on the parameter map by selecting the size of the sub-areas. Accordingly, the biomarker determined according to the disclosure as a single value can characterize the heterogeneity of the entire examination area with variable granularity.

In addition to the selected size of the sub-areas, i.e. the granularity, and the type of parameter map itself, the informative value of the biomarker determined according to the disclosure depends on the at least two parameter classes. In particular, the type of parameter map and the at least two parameter classes are preferably matched to one another such that the biomarker quantifies a disease and/or a course of therapy. A biomarker of this type is particularly informative. With the aid of a few settings, in particular the size of the sub-areas and the parameter classes provided, the biomarker can be determined in a highly reproducible and robust manner. In particular, the biomarker is an objective measure of homogeneity, which, in particular in the medical environment, enables a standardized appraisal of the parameter map independently of the visual perception of people and/or a radiologist.

One embodiment of the method provides that the further sub-areas that are at least partially different from one another merge into one another by displacement. The voxels of the parameter map are typically arranged in Cartesian fashion and the sub-area and the further sub-areas are preferably embodied as cuboid. The sub-area and the further sub-areas are preferably of the same size. A first further sub-area of the at least two further sub-areas can emerge from the sub-area in that the sub-area is shifted by at least one voxel in a Cartesian direction. A second further sub-area of the at least two further sub-areas can emerge from the first further sub-area in that the first further sub-area is shifted by at least one voxel in this Cartesian direction. If, for example, the sub-area and the at least two further sub-areas have a size of 5×5×5 voxels and the parameter map comprises 20×20×5 voxels, the parameter map can be optimally covered by 19×19 shifts of the sub-areas by one voxel in each case. It is also conceivable for a shift by the size of the sub-areas to take place. In this case, the parameter map would be covered by 4×4 shifts of the sub-areas by five voxels in each case. This embodiment enables particularly precise coverage of the parameter map by sub-areas. The statistical homogeneity value resulting therefrom is based on a large number of heterogeneity indicators as a result of which the biomarker is particularly robust.

One embodiment of the method additionally provides for the provision of a binary property map for the voxels indicating the presence of a property for each voxel, wherein only voxels having the property are taken into account when the sub-area is selected.

The provision of the binary property map can comprise the generation of the property map based on the parameter map. The property map can correlate with the parameter map. A property of a voxel can, for example, be defined by a parameter value of the voxel and/or by a parameter value of the voxel in a defined value range. The property map can be determined independently of the parameter map for the examination area. The property map can be based on medical image data and/or pathological data. The property map can indicate the presence of at least one of the following properties:
organ,
cells exhibiting a defined disease and/or change, in particular a tumor,
pathological change,
physical and/or physiological property,
voxels which have undergone a change in the course of longitudinal monitoring,
voxels with a parameter value according to the parameter map that is greater or smaller than a threshold value.

Accordingly, the property map can be referred to as a mask for the parameter map.

According to this embodiment, the selection of the sub-area preferably comprises a comparison of the parameter map with the property map. In the selection of the sub-area, preferably exclusively voxels having the property are taken into account. For this purpose, it is, for example, possible for a sub-area of a defined size to be superimposed on the parameter map, which is then restricted by superimposing the property map and exclusively comprises voxels for which the property map indicates the presence of the property, in particular voxels having the property.

This embodiment enables a more precise, in particular a focused determination of a biomarker. A biomarker determined in this way can quantify a homogeneity very selectively with respect to the property by the choice of property map. The use of the property map reduces noise in the data on which the biomarker is based, which is restricted to the relevant voxels having the property by the use of the property map. This makes the biomarker particularly precise and highly reproducible.

One embodiment of the method provides that the method steps D) and E) are only executed if the property map indicates the presence of the property for a minimum number of voxels within the sub-area, i.e. in particular, if the sub-area comprises the minimum number of voxels.

Accordingly, this embodiment provides that the determination of the frequency value for all parameter classes and the determination of the heterogeneity indicator from the frequency values for the selected sub-area only takes place when the sub-area comprises the minimum number of voxels and/or a minimum number of voxels of the sub-area has the property.

The minimum number of voxels can be specified relative to the number of voxels that the sub-area would have comprised before the restriction by the property map. The minimum number of voxels can, for example, be at least 20%, preferably at least 30%, particularly preferably at least 40% of the number of voxels that the sub-area would have comprised before the restriction by the property map. The minimum number of voxels depends in particular on the spatial resolution. The minimum number of voxels can, for example, be at least 5, preferably at least 10, particularly preferably at least 15. The minimum number of voxels can also be relative to the number of parameter classes provided. The minimum number of voxels can, for example, be at least twice the number of parameter classes provided.

This embodiment ensures that each heterogeneity indicator used to determine the statistical homogeneity value is informative and has a sufficiently high statistical significance. This increases the informative value and robustness of the statistical homogeneity value and thus of the biomarker.

One embodiment of the method provides that the heterogeneity indicator comprises a first heterogeneity indicator value corresponding to the largest frequency value of all frequency values of the parameter classes for the sub-area.

The first heterogeneity indicator value can also be referred to as a local homogeneity value. If, for example, three parameter classes are provided and, within the sub-area, 20% of the voxels, i.e. the parameter values, are to be assigned to the first parameter class, 35% to the second parameter class and 45% to the third parameter class, the corresponding frequency values are 20%, 35% and 45%. In this example, the first heterogeneity indicator value is 45%. The greater the local homogeneity, i.e. the more voxels, i.e. parameter values, within a sub-area are to be assigned to a single parameter class, the greater the first heterogeneity indicator value. If the biomarker comprises a first statistical homogeneity value based on the first heterogeneity indicator value, local inhomogeneities distributed over the parameter map can be quantified particularly well.

One embodiment of the method provides that the heterogeneity indicator comprises a second heterogeneity indicator value, said second heterogeneity indicator value assuming a first value if the frequency values of at least two parameter classes in each case exceed a threshold value and otherwise the second heterogeneity indicator value assumes a second value.

The second heterogeneity indicator value can also be referred to as a local discordance. The second heterogeneity indicator value can be a measure of a uniform distribution of the parameter values among the parameter classes. For example, three parameter classes are provided and, in the first sub-area, the frequency value is 20% for the first parameter class, 35% for the second parameter class and 45% for the third parameter class, wherein 20% is defined as the threshold value for the first and third parameter class. In this example, the second heterogeneity indicator value assumes the first value, for example 100. In a second and third sub-area, the frequency value may be 40% for the first parameter class, 50% for the second parameter class and 10% for the third parameter class. There, the first heterogeneity indicator value in each case assumes the second value, for example 0. When forming the second statistical homogeneity value based on these three second heterogeneity indicator values, a second statistical homogeneity value of 33 results when the mean value is calculated. If the biomarker comprises a second statistical homogeneity value based on the second heterogeneity indicator value, this can in particular control a measure of the distribution of the parameter values among the parameter classes weighted according to the threshold values. The biomarker can comprise a first statistical homogeneity value and a second statistical homogeneity value.

One embodiment of the method provides that the threshold values for the at least two parameter classes differ by at most 20%. The threshold values for the at least two parameter classes preferably differ by at most 10%, particularly preferably by at most 5%. The threshold values for the at least two parameter classes can also be of the same size. A second statistical homogeneity value and/or second heterogeneity indicator value determined in this way are particularly sensitive with respect to a uniform distribution of the parameter values among the parameter classes.

One embodiment of the method additionally comprises the establishment of a diagnosis and/or therapy monitoring based on the biomarker. In particular, a recommendation for therapy and/or an appraisal of the efficiency of the therapy performed can be ascertained based on the biomarker. Depending upon the parameter map, the biomarker can also diagnose a disease. Such a diagnosis and/or appraisal and/or recommendation can be automatically established and provided.

One embodiment of the method provides that the parameter map is based on magnetic resonance data and comprises at least one of the following values in spatial resolution:
ADC,
T1 relaxation time,
T2 relaxation time,
T2* relaxation time,
proton density,
perfusion parameter, in particular flow rate and/or permeability,
elasticity parameter,
fat content and/or fat percentage.

Magnetic resonance data is typically recorded from the examination area of a subject of the examination with a magnetic resonance device. Depending on the MR control sequence used for the recording, the T1 relaxation time (T1), the T2 relaxation time (T2), the proton density, the flow rate of a liquid and/or the apparent diffusion coefficient (ADC) can be extracted as quantitative parameter maps from the magnetic resonance data. Such quantitative parameter maps are in particular suitable for longitudinal monitoring of a pathological change and/or the assessment of the efficiency of the therapy.

One embodiment of the method provides that at least three parameter classes are provided and each of the three parameter classes indicates a stage of a tumor.

The first of the three parameter classes can comprise parameter values representative of active tumor cells. The third of the three parameter classes can comprise parameter values representative of dead cells. The second of the three parameter classes can comprise parameter values representative of an ambiguous state of the cells. In the case of tumors, heterogeneity correlates with malignancy and with tumor response to chemotherapy. Consequently, this embodiment of the method enables a particularly good quantitative evaluation and appraisal of malignancy and tumor response to chemotherapy. Conventionally, parameter maps are evaluated purely visually by a radiologist for this purpose. The biomarker enables independent and objective appraisal.

One embodiment of the method provides that the parameter map is an ADC map, at least three parameter classes are provided and each of the three parameter classes indicates a stage of a tumor, the first parameter class of the three parameter classes comprises parameter values up to a maximum of 800 and the third parameter class of the three parameter classes comprises parameter values of at least 950.

The first parameter class comprises parameter values, i.e. ADC values, preferably of at most 750, particularly preferably of at most 700, and/or is representative of active tumor cells. The third parameter class comprises parameter values, i.e. ADC values, preferably of at least 1000 and/or is representative of dead tumor cells and/or successful radiotherapy and/or successful chemotherapy. The second parameter class preferably comprises the ADC values between the first and the third parameter class. Further parameter classes are conceivable.

In this embodiment, a first statistical homogeneity value based on the first heterogeneity indicator value correlates with the spatial homogeneity of the effectiveness of the treatment. If the treatment is uniformly effective over the examination area, the first statistical homogeneity value is approximately 100%. If the effectiveness is heterogeneous, it is in the range between 30% and 50%.

In this embodiment, a second statistical homogeneity value based on the second heterogeneity indicator value correlates with the spatial heterogeneity of the effectiveness of the treatment. If the second heterogeneity indicator value is determined by the fact that it assumes 100 as the first value if the frequency values exceed a threshold value of 20% in at least two parameter classes and otherwise assumes 0 as the second value 0, the more heterogeneously the treatment works, the closer the second heterogeneity indicator value is to 100.

The quantitative appraisal of the effectiveness of cancer therapy by means of the biomarker enables individual and also local adaptation of the treatment.

One embodiment of the method provides that the parameter map is based on medical image data, in particular on magnetic resonance (MR) data and/or computed tomography (CT) data and/or positron emission tomography (PET) data. Magnetic resonance data and/or computed tomography data and/or PET data can comprise spatially resolved quantitative data that can be used directly as a parameter map. The gray values of image data can correspond to parameter values. In particular in the medical area, the homogeneity of parameter maps may be indicative of a person's health and/or a disease. Accordingly, a biomarker determined in this way is particularly valuable.

Furthermore, the disclosure is based on an image evaluator with an ascertaining unit comprising an input, an output and a determining unit. The ascertaining unit is embodied to execute a method according to the disclosure for generating a biomarker.

The ascertaining unit can be provided with a medical parameter map, medical image data, at least one parameter class, information with respect to the size and/or shape of the sub-area, a property map and/or a threshold value for a frequency value via the input. Further functions, algorithms or parameters required in the method can be provided to the ascertaining unit via the input. The statistical homogeneity value as a biomarker and/or heterogeneity indicators and/or first and/or second heterogeneity indicator values and/or a diagnosis and/or result of therapy monitoring and/or further results of an embodiment of the method according to the disclosure can be provided via the output. The ascertaining unit can be integrated in the image evaluator. The ascertaining unit can also be installed separately from the image evaluator. The ascertaining unit can be connected to the image evaluator.

Embodiments of the image evaluator according to the disclosure are embodied similarly to the embodiments of the method according to the disclosure. The image evaluator can have further control components which are necessary and/or advantageous for executing a method according to the disclosure. The image evaluator can also be embodied to send control signals and/or to receive and/or process control signals in order to execute a method according to the disclosure. Computer programs and further software by means of which the processor unit of the ascertaining unit automatically controls and/or executes a method sequence of a method according to the disclosure can be stored on a memory unit of the ascertaining unit.

A computer program product according to the disclosure can be loaded directly into a memory unit of a programmable ascertaining unit and has program code means for executing a method according to the disclosure when the computer program product is executed in the ascertaining unit. This enables the method according to the disclosure to be executed quickly, identically repeatedly and robustly. The computer program product is configured such that it can execute the method steps according to the disclosure by means of the ascertaining unit. Herein, the ascertaining unit must in each case fulfil the requisite conditions, for example, having an appropriate random access memory, an appropriate graphics card or an appropriate logic unit so that the respective method steps can be executed efficiently. The computer program product is, for example, stored on an electronically readable medium or held on a network or server from where it can be loaded into the processor of a local ascertaining unit. Examples of electronically readable data carriers are a DVD, a magnetic tape or a USB stick on which electronically readable control information, in particular software, is stored. When this control information (software) is read from the data carrier and stored in an ascertaining unit, all the embodiments according to the disclosure of the above-described method can be performed.

Furthermore, the disclosure is based on an electronically readable data carrier on which a program is held and which is provided to execute a method for generating a biomarker.

The advantages of the inventive image evaluator, the inventive computer program product and the inventive electronically readable data carrier substantially correspond to the advantages of the method according to the disclosure for generating a biomarker which are described in detail above. Features, advantages or alternative embodiments can likewise also be transferred to the other claimed subject matter and vice versa.

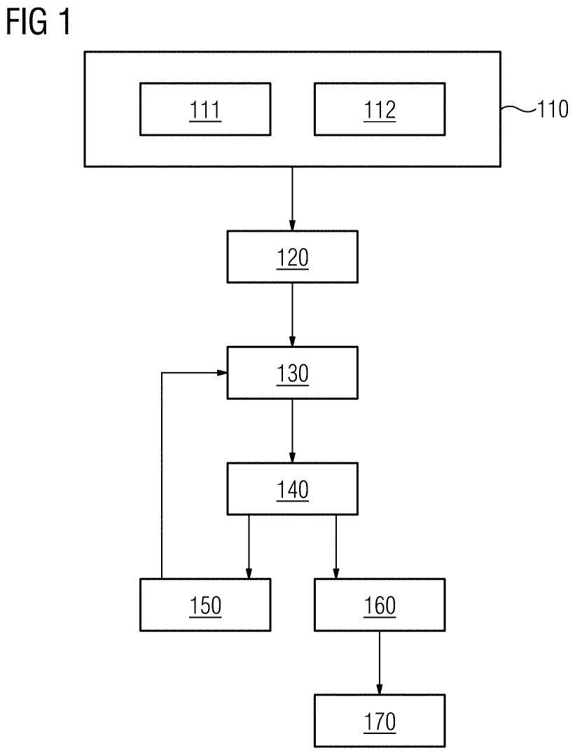

FIG. 1 shows a flow diagram of a first embodiment of a method according to the disclosure. The method according to the disclosure for generating a biomarker quantifying spatial homogeneity of a medical parameter map starts with method step 110, the provision of data. In particular method step 110, i.e. the provision of data, comprises method step 111, the provision of a medical parameter map, wherein the parameter map in each case has a parameter value for a plurality of voxels 52 mapping an examination area. Likewise, method step 110, i.e. the provision of data, comprises method step 112, the provision of at least two parameter classes, wherein each parameter value is to be assigned to a parameter class. The following method step 120 comprises the selection of a sub-area of the examination area. Method step 130 comprises the determination of a frequency value for each parameter class for the sub-area, said frequency value indicating the proportion of the parameter values occurring in this parameter class in the sub-area. Method step 140 comprises the determination of a heterogeneity indicator for the sub-area taking into account the frequency values of the parameter classes. In method step 150, at least two further sub-areas are selected with the method steps 130 and 140 being executed for said at least two further sub-areas. Herein, method step 150 can in particular be executed before, after or simultaneously with method step 140, i.e. the determination of a heterogeneity indicator for the sub-area. In particular, for each sub-area and the at least two further sub-areas, the frequency values can first be determined for each parameter class before a heterogeneity indicator is determined according to method step 140 for each sub-area. Method step 160 comprises a statistical evaluation of the heterogeneity indicator of the sub-area and the further heterogeneity indicators of the further sub-areas and thus the ascertaining of a statistical homogeneity value. In method step 170, the statistical homogeneity value is provided as a biomarker.

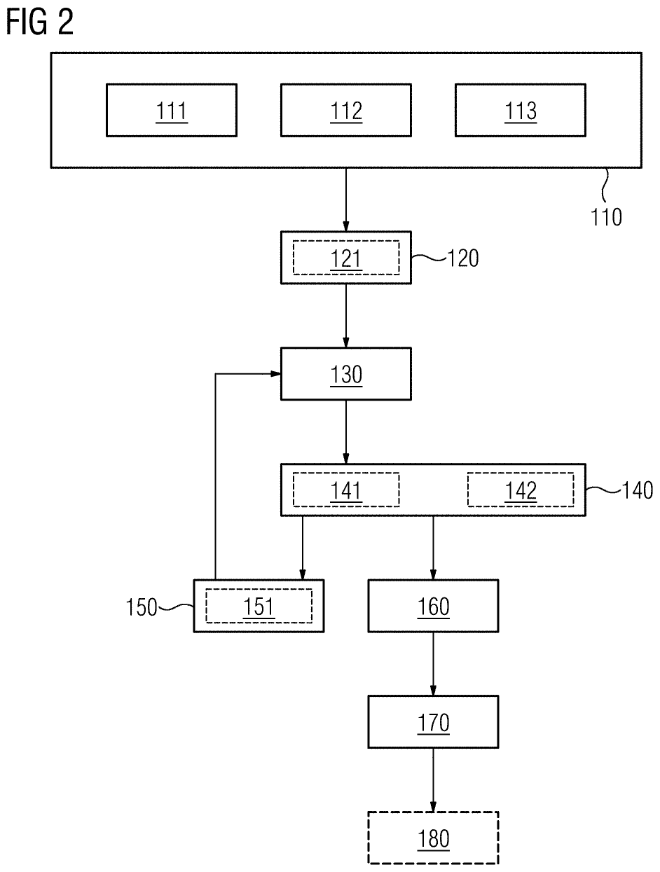

FIG. 2 shows a flow diagram of a second embodiment of a method according to the disclosure. The second embodiment of the method according to the disclosure differs from the first embodiment shown in FIG. 1 in the following method steps. The method step 110, the provision of the data, comprises, with method step 113, the provision of a binary property map. In particular, method step 120, the selection of a sub-area, can, with method step 121, comprise a check, with, according to said check, the method steps 130, 140 and 150 only being executed for the sub-area if the property map indicates the presence of the property for a minimum number of voxels 52 within the sub-area.

Method step 140, the determination of a heterogeneity indicator, can comprise method step 141, the determination of a first heterogeneity indicator value, and/or method step 142, the determination of a second heterogeneity indicator value. In particular, the heterogeneity indicator can comprise the first heterogeneity indicator value and/or second heterogeneity indicator value, which is taken into account with the statistical homogeneity value in method step 160. In particular, the statistical homogeneity value can comprise a first statistical homogeneity value based on statistical evaluation of the first heterogeneity indicator value of the sub-area and the further first heterogeneity indicator values of the further sub-areas. Alternatively and/or additionally to the first statistical homogeneity value, the statistical homogeneity value can comprise a second statistical homogeneity value based on statistical evaluation of the second heterogeneity indicator value of the sub-area and the further second heterogeneity indicator values of the further sub-areas. Optionally, in method step 180, a diagnosis can be established and/or therapy monitoring can take place based on the biomarker.

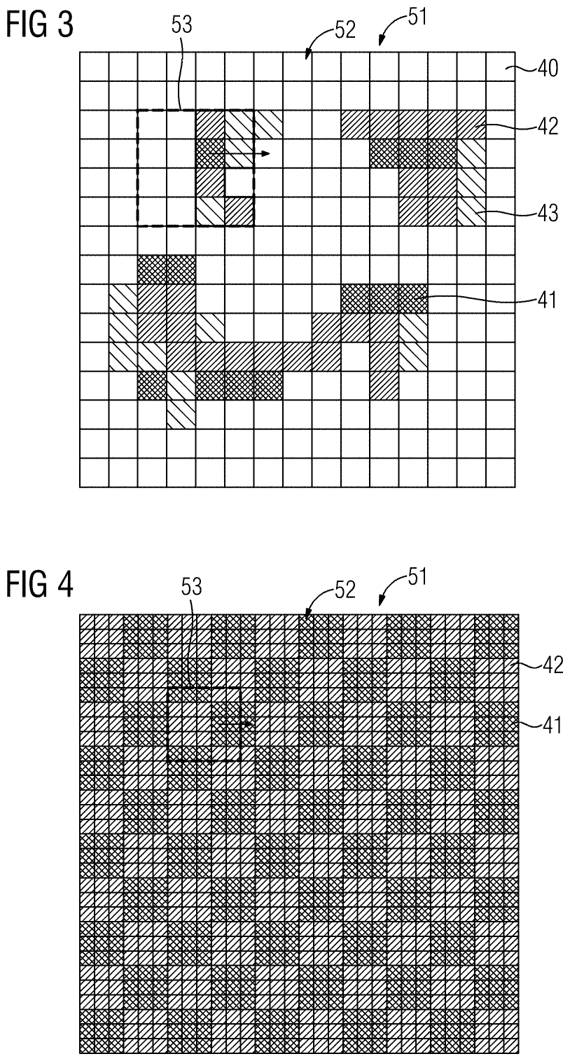

FIG. 3 shows a parameter map of the examination area 51 after the parameter values have in each case been assigned to one parameter class of three parameter classes 41, 42, 43 superimposed by a property map and the selection of a sub-area 53 using a binary property map in a schematic depiction.

The examination area 51 comprising a large number of voxels 52 is depicted schematically.

A property map is depicted superimposed on the examination area 51, said property map dividing the examination area 51 into voxels 52 having the property 41, 42, 43 and voxels 52 not having the property 40. On the selection of the sub-area 53, first a defined number of voxels, in the case depicted 4x4 voxels within the slice depicted, are selected as sub-area 53, wherein only the voxels 52 having the property 41, 42, 43 are taken into account for the sub-area 53. Consequently, in the case depicted, the sub-area 53 only comprises a fraction of the 4x4 voxels within the slice depicted. If the embodiment of the method according to the disclosure provides for a check according to method step 121, according to which, for example, at least eight voxels must have the property in order for frequency values to be determined for the sub-area 53, this would not be fulfilled in the present case, since only seven voxels have the property. A further sub-area 53 can emerge according to method steps 150, 151 by shifting the 4×4 voxels shown by one column to the right along the arrow. This shift is also known as the “sliding window technique”. In this case, the further sub-area 53 would comprise eight voxels having the property. Hence, frequency values would be determined for the further sub-area 53.

In this case, the parameter map can be based on magnetic resonance data and embodied as an ADC map. Herein, the three parameter classes 41, 42, 43 can each indicate a stage of a tumor. Thus, the first parameter class 41 can, for example, comprise ADC values below 700, the second parameter class 42 ADC values from 700 to 1000 and the third parameter class 43 ADC values above 1000. Herein, the first parameter class 41 can indicate voxels 52 with active tumor cells. The third parameter class 43 can indicate voxels 52 with dead cells, in particular cells in which cancer therapy was successful. The second parameter class 42 can indicate cells with an unclear therapy response.

FIG. 4 shows an example for ascertaining a statistical homogeneity value for a first parameter map after the parameter values have in each case been assigned to one parameter class of two parameter classes 41, 42. This assignment is a checkerboard-like arrangement of the parameter classes 41, 42 with a size of 3×3 voxels in each case. 5×5 voxels are selected as sub-area 53 and the sub-area 53 is shifted iteratively and voxel-by-voxel over the entire examination area 51 in the context of the method for generating the further sub-areas.

In the case depicted, the first statistical homogeneity value, based on a mean value of the first heterogeneity indicator values of the sub-areas each corresponding to the greatest frequency value of all the frequency values of the parameter classes of the respective sub-area 53, is 52.4%. This indicates that on average more than half of the voxels 52 in the sub-area 53 are to be assigned to the same parameter class.

Let a second heterogeneity indicator value be determined by the fact that it assumes 100 as the first value if the frequency values of both parameter classes 41, 42 in each case exceed a threshold value of 20% and otherwise assumes 0 as the second value 0. Then, in the case depicted, the second statistical homogeneity value based on a mean value of the second heterogeneity indicator values is 99.9. This high second statistical homogeneity value indicates that in almost no window of the size 5×5 there are fewer than 20% voxels in the first parameter class 41 or the second parameter class 42.

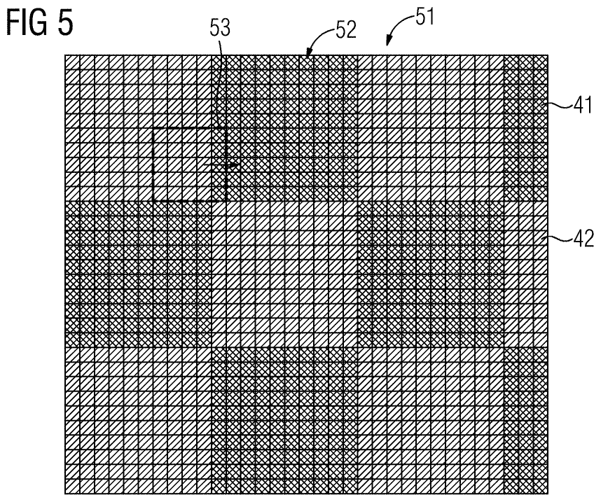

FIG. 5 shows an example for ascertaining a statistical homogeneity value for a second parameter map after the parameter values have in each case been assigned to one parameter class of two parameter classes 41, 42. This assignment is a checkerboard-like arrangement of the parameter classes 41, 42 with in each case a size of 10×10 voxels. 5×5 voxels are selected as sub-area 53 and the sub-area 53 is shifted iteratively and voxel-by-voxel over the entire examination area 51 in the context of the method for generating the further sub-areas.

In the case depicted, the first statistical homogeneity value based on a mean value of the first heterogeneity indicator values of the sub-areas each corresponding to the greatest frequency value of all the frequency values of the parameter classes of the respective sub-area 53, is 80.0.

Let a second heterogeneity indicator value be determined by the fact that it assumes 100 as the first value if the frequency values of both parameter classes 41, 42 in each case exceed a threshold value of 20% and otherwise assumes 0 as the second value. Then, in the case depicted, the second statistical homogeneity value based on a mean value of the second heterogeneity indicator values is 61.3.

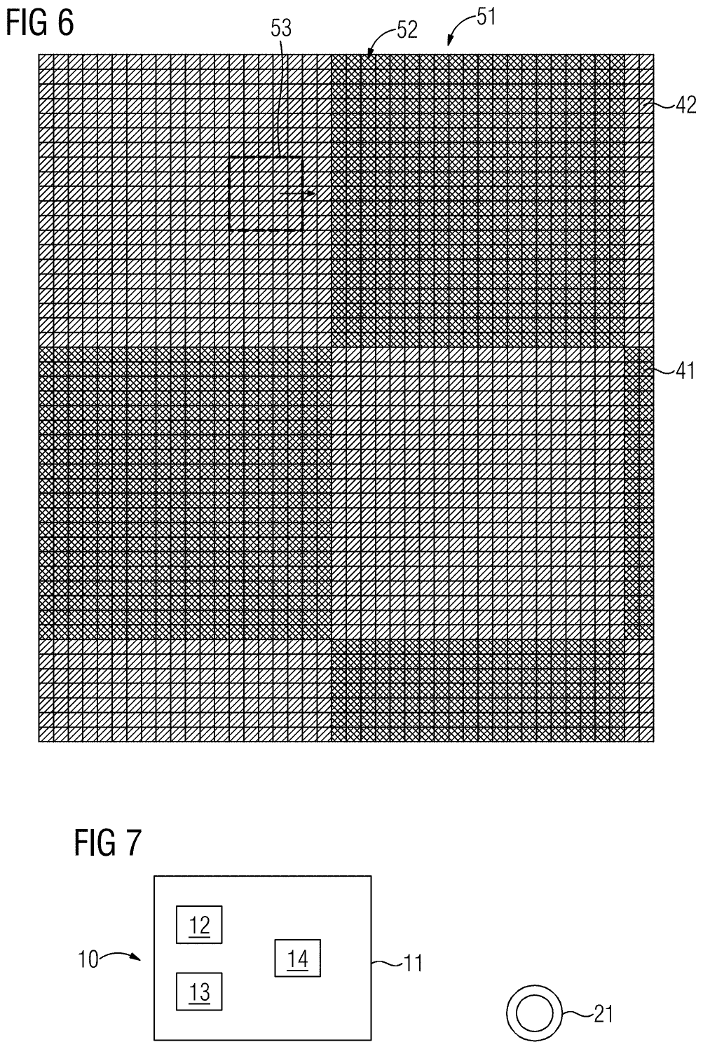

FIG. 6 shows an example for ascertaining a statistical homogeneity value for a third parameter map after the parameter values have in each case been assigned to one parameter class of two parameter classes 41, 42. This assignment is a checkerboard-like arrangement of the parameter classes 41, 42 with in each case a size of 20×20 voxels. 5×5 voxels are selected as sub-area 53 and the sub-area 53 is shifted iteratively and voxel-by-voxel over the entire examination area 51 in the context of the method for generating the further sub-areas.

In the case depicted, the first statistical homogeneity value based on a mean value of the first heterogeneity indicator values of the sub-areas each corresponding to the greatest frequency value of all the frequency values of the parameter classes of the respective sub-area 53, is 89.9. This value indicates a high degree of homogeneity since the majority of the voxels 52 within the sub-area 53 belong to one parameter class.

Let a second heterogeneity indicator value be determined by the fact that it assumes 100 as the first value if the frequency values of both parameter classes 41, 42 in each case exceed a threshold value of 20% and otherwise assumes 0 as the second value. Then, in the case depicted, the second statistical homogeneity value based on a mean value of the second heterogeneity indicator values is 33.0. This low second statistical homogeneity value indicates that only a few of the sub-areas have a balanced ratio of the two parameter classes 41, 42.

FIG. 7 shows an image evaluator 10 comprising an ascertaining unit 11 for executing a method according to the disclosure for generating a biomarker quantifying spatial homogeneity in a schematic depiction. The ascertaining unit 11 may comprises a determining unit (determiner) 14, an input 12, and an output 13. The ascertaining unit 11 may be referred to as biomarker generator 11 in one or more aspects. In an exemplary embodiment, the evaluator 10 includes processing circuitry that is configured to perform one or more functions and/or operations of the evaluator 10. In this example, one or more of the components of the evaluator 10 may additionally or alternatively include processing circuitry that is configured to perform one or more of the functions of the respect component(s). In an exemplary embodiment, the evaluator 10 is additionally configured to control the operation of an imaging device, such as the imaging device that provides medical imaging data that is processed by the evaluator 10. In this example, the evaluator 10 may be referred to as a controller.

The ascertaining unit 11 is in addition configured to execute a method for generating a biomarker quantifying spatial homogeneity. For this purpose, the ascertaining unit 11 has computer programs and/or software, which can be loaded directly into a memory unit (not shown in further detail) of the ascertaining unit 11, with program means for executing a method for generating a biomarker quantifying spatial homogeneity when the computer programs and/or software are executed in the ascertaining unit 11. For this purpose, the ascertaining unit 11 has a processor (not shown in further detail), which is adapted to execute the computer programs and/or software. Alternatively, the computer programs and/or software can also be stored on an electronically readable data carrier 21 embodied as separate from the image evaluator 10 and/or ascertaining unit 11, wherein data access from the ascertaining unit 11 to the electronically readable data carrier 21 can take place via a data network.

A method for generating a biomarker quantifying spatial homogeneity can also be present in the form of a computer program product that implements the method on the generating unit 11 when it is executed on the generating unit 11. Likewise, an electronically readable data carrier 21 with electronically readable control information which comprises at least one computer program product as described above is embodied such that it performs the described method when the data carrier 21 is used in a generating unit 11 of an image evaluator 10.

Although the disclosure has been illustrated and described in detail by the preferred exemplary embodiments, the disclosure is not restricted by the examples disclosed and other variations can be derived herefrom by the person skilled in the art without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for generating a biomarker quantifying spatial homogeneity of a medical parameter map, wherein the parameter map in each case has a parameter value for a plurality of voxels mapping an examination area, the method comprising:
A) providing the parameter map;
B) providing at least two parameter classes, wherein each parameter value is to be assigned to a parameter class;
C) selecting a sub-area of the examination area;
D) determining, in each case, a frequency value for each parameter class for the sub-area, the frequency value indicating a proportion of the parameter values occurring the respective parameter class in the sub-area;
E) determining a heterogeneity indicator for the sub-area based on the frequency values of the parameter classes;
F) repeating operations C), D), and E) for at least two further sub-areas to generate further heterogeneity indicators for the at least two further sub-areas, wherein the at least two further sub-areas are at least partially different from one another;
G) statistically evaluating the heterogeneity indicator of the sub-area and the further heterogeneity indicators of the further sub-areas to ascertain a statistical homogeneity value; and
H) providing the statistical homogeneity value as a biomarker.

2. The method as claimed in claim 1, wherein the further sub-areas that are at least partially different from one another merge into one another by displacement.

3. The method as claimed in claim 1, further comprising provisioning a binary property map for the voxels indicating a presence of a property for each voxel, wherein only voxels having the property are taken into account when the sub-area is selected.

4. The method as claimed in claim 3, wherein execution of operations D) and E) is omitted in response to the property map indicating the presence of the property for a minimum number of voxels within the sub-area.

5. The method as claimed in claim 1, wherein the heterogeneity indicator comprises a first heterogeneity indicator value corresponding to a greatest frequency value of all frequency values of the parameter classes for the sub-area.

6. The method as claimed in claim 5, wherein the heterogeneity indicator comprises a second heterogeneity indicator value whose value is set based on the frequency values of at least two parameter classes in each case and respect threshold values.

7. The method as claimed in claim 6, wherein the threshold values for the at least two parameter classes differ by at most 20%.

8. The method as claimed in claim 1, further comprising establishing a diagnosis and/or therapy monitoring based on the biomarker.

9. The method as claimed in claim 1, wherein at least three parameter classes are provided and each of the three parameter classes indicates a stage of a tumor.

10. The method as claimed in claim 1, wherein the parameter map comprises at least one of the following values in spatial resolution:
ADC,
T1 relaxation time,
T2 relaxation time,
T2* relaxation time,
proton density,
perfusion parameter, in particular flow rate and/or permeability,
elasticity parameter, and
fat content and/or fat percentage.

11. The method as claimed in claim 1, wherein:
the parameter map is an apparent diffusion coefficient (ADC) map,
at least three parameter classes are provided and each of the three parameter classes indicates a stage of a tumor, and
the first parameter class of the three parameter classes comprises parameter values up to a maximum of 800 and the third parameter class of the three parameter classes comprises parameter values of at least 950.

12. The method as claimed in claim 1, wherein the parameter map is based on medical image data that includes: magnetic resonance (MR) data, computed tomography (CT) data, and/or positron emission tomography (PET) data.

13. The method as claimed in claim 1, further comprising providing the biomarker in electronic form as a data file.

14. A computer program product, which comprises a program and is loadable directly into a memory of a programmable processor, when the program is executed by the processor, controls the processor to generate a biomarker as claimed in claim 1.

15. A non-transitory computer-readable storage medium with an executable program stored thereon, that when executed, instructs a processor to perform the method of claim 1.

16. An image evaluator adapted to generate a biomarker quantifying spatial homogeneity of a medical parameter map, the parameter map in each case having a parameter value for a plurality of voxels mapping an examination area, the evaluator comprising:
a memory configured to store computer-readable instructions; and
processing circuitry configured to execute the computer-readable instructions stored in the memory to:
A) provide the parameter map;
B) provide at least two parameter classes, wherein each parameter value is to be assigned to a parameter class;
C) select a sub-area of the examination area;
D) determine, in each case, a frequency value for each parameter class for the sub-area, the frequency value indicating a proportion of the parameter values occurring the respective parameter class in the sub-area;
E) determine a heterogeneity indicator for the sub-area based on the frequency values of the parameter classes;
F) repeat operations C), D), and E) for at least two further sub-areas to generate further heterogeneity indicators for the at least two further sub-areas, wherein the at least two further sub-areas are at least partially different from one another;
G) statistically evaluate the heterogeneity indicator of the sub-area and the further heterogeneity indicators of the further sub-areas to ascertain a statistical homogeneity value; and
H) provide the statistical homogeneity value as a biomarker.
